(19) United States
(12) Patent Application Publication
(10) Pub. No.: US 2026/0263077 A1
(43) Pub. Date: Sep. 10, 2026

(54) DEVICE AND METHOD FOR VARIABLE BLOOD FLOW OCCLUSION

(71) Applicant: Cardio-Renal Solutions, Inc., San Francisco, CA (US)

(72) Inventor: Troy THORNTON, San Francisco, CA (US)

(21) Appl. No.: 19/166,447

(22) PCT Filed: Mar. 18, 2024
(86) PCT No.: PCT/US2024/020443

§ 371 Date: Sep. 17, 2025

Related U.S. Application Data
(60) Provisional application No. 63/490,936, filed on Mar. 17, 2023.

(51) Int. Cl. A61B 17/12

(52) U.S. Cl. CPC A61B 17/12109; A61B 17/12036; A61B 2017/1205

(57) ABSTRACT

A blood flow control device with a catheter advanceable into a blood vessel to a blood flow control site; an anchor having an uncompressed configuration and a compressed configuration, the anchor comprising a proximal portion and a distal portion with a smaller diameter than the proximal portion, the proximal portion expandable toward the uncompressed configuration to engage a wall of the blood vessel, a blood impermeable wall defines a blood flow path through the anchor from a proximal opening to a distal opening at a distal end of the distal portion, the catheter being disposed outside of the adjustable blood flow path; a flow element comprising a cinching line to change a shape of the distal portion of the anchor and a rate of blood flow through the blood flow path when the distal portion of the anchor changes from a first shape to a second shape.

### PRIORITY CLAIM

This patent application claims priority to U.S. provisional patent application no. 63/490,936, titled “DEVICE AND METHOD FOR VARIABLE BLOOD FLOW OCCLUSION,” and filed on Mar. 17, 2023, which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

In patients with heart failure, reduced cardiac output can lead to reduced renal perfusion, which in turn can cause decreased urine output, activation of the sympathetic nervous system, and other neurohormonal changes. These compensatory mechanisms may occur in an attempt by the body to increase the blood volume to help maintain cardiac output. However, the increased blood volume can be detrimental, particularly because it increases venous pressure. The increased venous pressure can cause, for example, pulmonary and systemic edema or venous congestion. Higher venous pressure can also make it more difficult for the kidneys to function to remove fluid.

Increased venous pressure, or increased preload on the right heart, is thus detrimental to the recovery of heart failure patients. Accordingly, decreasing preload on the right heart is desired, as decreased preload lowers the cardiac filling pressure and increases cardiac output. Decreased venous pressure can also decrease renal vein pressure, increasing the pressure differential across the kidneys, which may enhance diuresis of the patient.

Many patients in heart failure are treated with diuretic drugs to reduce blood volume and venous pressure in order to reduce edema, but diuretic drugs are frequently ineffective, especially in patients with renal dysfunction or diuretic resistance. Device-based therapies have also been proposed, including devices that expand within a blood vessel, such as the inferior vena cava (IVC) or the superior vena cava (SVC), to partially occlude the blood vessel for an extended time or to completely occlude the blood vessel for a short time.

US Pat. Publ. No. 2022/0287831 describes a catheter-based blood flow control device that can be placed either in the superior vena cava (SVC) to decrease blood flow returning from the head and upper extremities or in the inferior vena cava (IVC) in an infrarenal location in order to decrease blood flow returning from the lower extremities. The device can restrict blood flowing in the antegrade direction, resulting in a higher pressure on the inflow (proximal) side and a lower pressure on the outflow (distal) side.

### SUMMARY OF THE DISCLOSURE

Described herein are catheter-based blood flow control devices that can be placed either in the superior vena cava (SVC) to decrease blood flow returning from the head and upper extremities or in the inferior vena cava (IVC) in an infrarenal location in order to decrease blood flow returning from the lower extremities. The devices can restrict blood flowing in the antegrade direction, resulting in a higher pressure on the inflow (proximal) side and a lower pressure on the outflow (distal) side.

In general, a blood flow control device can comprise a catheter adapted to be advanced into a blood vessel to a blood flow control site within the blood vessel; an anchor supported by the catheter and having an uncompressed configuration and a compressed configuration, the anchor comprising a proximal portion having first diameter in the uncompressed configured and a distal portion having a second diameter in the uncompressed configuration smaller than the first diameter, the proximal portion being adapted to expand toward the uncompressed configuration to engage a wall of the blood vessel, the anchor comprising a blood impermeable wall defining an adjustable blood flow path extending through the anchor from a proximal opening at a proximal end of the proximal portion to a distal opening at a distal end of the distal portion, the catheter being disposed outside of the adjustable blood flow path; a flow control element supported by the catheter, the flow control element comprising a cinching line adapted to change a shape of the distal portion of the anchor from a first shape to a second shape, thereby changing a size of the distal opening, the cinching line being disposed proximal to the distal end of the distal portion and being adapted to change a rate of blood flow through the blood flow path when the distal portion of the anchor changes from the first shape to the second shape; and a blood flow control actuator disposed at a proximal section of the catheter and adapted to actuate the cinching line.

In this and other examples, a blood flow control device may also comprise one or more of the following. The cinching line can extend around an exterior surface of the anchor. The cinching line can extend through a plurality of supports circumferentially disposed around the distal portion of the anchor. Each of the plurality of supports can comprise a loop extending through the blood impermeable wall and around a strut of the anchor. The distal portion of the anchor may be further adapted such that its distal end does not bend radially inward with respect to remaining portions of the distal portion when the distal portion changes from the first shape to the second shape. The device can further comprise an anchor distal support adapted to resist movement of the distal portion of the anchor toward the catheter during actuation of the cinching line. The anchor can comprise a self-expandable scaffold having a plurality of struts, the distal support comprising a distal support strut that is one of the plurality of struts, the distal support strut being stiffer than other struts of the plurality of struts. The distal support strut can be disposed on a portion of the scaffold facing the catheter. The distal support strut can have an opening through which the cinching line extends. The device may further comprise a centering device configured to align the anchor with an opening of a sheath to facilitate placement of the anchor in the sheath. The anchor can be supported by the catheter on an exterior side of the catheter such that the catheter is outside of the anchor, the centering device comprising a ramped surface on the exterior side of the catheter. The device can further comprise an anchor collapse control element supported by the catheter and adapted to reduce a cross-sectional dimension of a proximal end of the anchor to facilitate placement of the anchor in the sheath. The anchor can comprise a scaffold comprising a plurality of rows of expandable cells defined by struts, cells in a distal row of cells of the plurality of rows of expandable cells having longer struts than cells in a plurality of more proximal rows of cells. The cells in the distal row of cells can extend over a larger area in the uncompressed configuration than areas over which the cells in the more proximal rows extend in the uncompressed configuration.

In general, a blood flow control device can comprise a catheter adapted to be advanced into a blood vessel to a blood flow control site within the blood vessel; an anchor supported by the catheter and having an uncompressed configuration and a compressed configuration, the anchor comprising a proximal portion having first diameter in the uncompressed configured and a distal portion having a second diameter in the uncompressed configuration smaller than the first diameter, the proximal portion being adapted to expand toward the uncompressed configuration to engage a wall of the blood vessel, the anchor comprising a blood impermeable wall defining an adjustable blood flow path extending through the anchor from a proximal opening at a proximal end of the proximal portion to a distal opening at a distal end of the distal portion, the catheter being disposed outside of the adjustable blood flow path; a flow control element supported by the catheter, the flow control element comprising a cinching line adapted to change a shape of the distal portion of the anchor from a first shape to a second shape, the cinching line extending to the anchor from the catheter and being adapted to change a rate of blood flow through the blood flow path when the distal portion of the anchor changes from the first shape to the second shape; an anchor distal support adapted to resist movement of the distal portion of the anchor toward the catheter during actuation of the cinching line; and a blood flow control actuator disposed at a proximal section of the catheter and adapted to actuate the cinching line.

In this and other examples, a blood flow control device may also comprise one or more of the following. The anchor can comprise a self-expandable scaffold having a plurality of struts, the distal support comprising a distal support strut that is one of the plurality of struts, the distal support strut being stiffer than other struts of the plurality of struts. The distal support strut can be disposed on a portion of the scaffold facing the catheter. The distal support strut can have an opening through which the cinching line extends. The distal portion of the anchor can be further adapted such that its distal end does not bend radially inward with respect to remaining portions of the distal portion when the distal portion changes from the first shape to the second shape. The cinching line can be further adapted to change a size of the distal opening when it changes the shape of the distal portion of the anchor.

In general, a blood flow control device can comprise a catheter adapted to be advanced into a blood vessel to a blood flow control site within the blood vessel; an anchor supported by the catheter on an exterior side of the catheter such that the catheter is outside of the anchor, the anchor having an uncompressed configuration and a compressed configuration, the anchor being adapted to expand toward the uncompressed configuration to engage a wall of the blood vessel, the anchor comprising a blood impermeable wall defining an adjustable blood flow path extending through the anchor from a proximal opening to a distal opening, the catheter being disposed outside of the adjustable blood flow path; a flow control element supported by the catheter, the flow control element being adapted to change a dimension of the adjustable blood flow path to change a rate of blood flow through the blood flow path, a centering device configured to align the anchor with an opening of a sheath to facilitate placement of the anchor in the sheath; and a blood flow control actuator disposed at a proximal section of the catheter and adapted to actuate the flow control element.

In this and other examples, a blood flow control device may also comprise one or more of the following. The anchor can be supported by the catheter on an exterior side of the catheter such that the catheter is outside of the anchor, the centering device comprising a ramped surface on the exterior side of the catheter. The device may further comprise an anchor collapse control element supported by the catheter and adapted to reduce a cross-sectional dimension of a proximal end of the anchor to facilitate placement of the anchor in the sheath.

In general, a blood flow control device can comprise a catheter adapted to be advanced into a blood vessel to a blood flow control site within the blood vessel; an anchor supported by the catheter on an exterior side of the catheter such that the catheter is outside of the anchor, the anchor having an uncompressed configuration and a compressed configuration, the anchor being adapted to expand toward the uncompressed configuration to engage a wall of the blood vessel, the anchor comprising a scaffold and a blood impermeable wall defining an adjustable blood flow path extending through the anchor from a proximal opening to a distal opening, the scaffold comprising a plurality of rows of expandable cells defined by struts, cells in a distal row of cells of the plurality of rows of expandable cells having longer struts than cells in a more proximal rows of cells, the catheter being disposed outside of the adjustable blood flow path; a flow control element supported by the catheter, the flow control element being adapted to change a dimension of the adjustable blood flow path to change a rate of blood flow through the blood flow path; and a blood flow control actuator disposed at a proximal section of the catheter and adapted to actuate the flow control element.

In some aspects, a blood flow control device is provided comprising: a catheter adapted to be advanced into a blood vessel to a blood flow control site within the blood vessel; an expandable anchor supported by the catheter, the expandable anchor being adapted to expand to engage a wall of the blood vessel, the expandable anchor comprising a blood impermeable wall defining an adjustable blood flow path extending through the expandable anchor from a proximal opening to a distal opening, the catheter being disposed outside of the adjustable blood flow path and defining at least one gap between an exterior of the blood impermeable wall and the blood vessel; and at least one expandable element disposed along a portion of the catheter and the expandable anchor, the at least one expandable element being configured to expand into the at least one gap to prevent blood from passing therethrough.

In some aspects, the at least one expandable element comprises one or more inflatable balloons.

In some aspects, an inflation lumen is disposed within the catheter, the inflation lumen being fluidly coupled to the one or more inflatable balloons.

In one aspect, the at least one expandable element comprises excess material disposed within apices of the anchor on an inflow end of the anchor, the excess material being configured to expand outwards to a larger diameter than that of the anchor when blood flows through the anchor.

In other aspects, one or more additional supports between the apices configured to expand outwards towards the blood vessel to cause the excess material to expand into the gap.

In some aspects, the at least one expandable element comprises excess material disposed a cell of the anchor that spans the catheter, the excess material being configured to expand outwards to a larger diameter than that of the anchor when blood flows through the anchor.

In one aspect, the at least one expandable element comprises excess material disposed at least two cells of the anchor on either side of the catheter, the excess material being configured to expand outwards to a larger diameter than that of the anchor when blood flows through the anchor.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a side elevational view of an anchor of an exemplary blood flow control device.

FIG. 2A is a bottom elevation view of the blood flow control device anchor of FIG. 1.

FIG. 2B is a detailed view of a distal end of the blood flow control device anchor of FIG. 1.

FIG. 3 is a side elevational view showing the anchor of FIG. 1 mounted on a catheter.

FIG. 4A is a side elevational view of the blood flow control device anchor of FIG. 1 with its distal opening in a more open configuration.

FIG. 4B is a side elevational view of the blood flow control device anchor of FIG. 1 is a side elevational view of the blood flow control device anchor of FIG. 1 with its distal opening in a more closed configuration.

FIG. 5A is a perspective view of the blood flow control device anchor of FIG. 1 with its distal opening in a more open configuration.

FIG. 5B is a perspective view of the blood flow control device anchor of FIG. 1 with its distal opening in a more closed configuration.

FIGS. 6A-6D show the blood flow control device of this invention being compressed and retracted into a sheath.

FIG. 7A is a flattened view of a cut pattern for forming the stent of the anchor of FIG. 1.

FIG. 7B is a side view of a cut tube forming the stent of the anchor of FIG. 1.

FIG. 7C is a flattened view of a cut pattern for forming the stent of the anchor of FIG. 1.

FIG. 7D is another example of a flattened view of a cut pattern for forming the stent of the anchor.

FIG. 8A is a cross-sectional view of the catheter of the blood flow control device taken along the line A-A in FIG. 3.

FIG. 8B is a cross-sectional view of the catheter of the blood flow control device taken along the line B-B in FIG. 3.

FIG. 9 is an example of the blood flow control device positioned in a vessel to illustrate impact on blood flow rates.

FIGS. 10A-10C are examples of cinch line engagement with the stent to adjust the struts and blood flow control device geometry.

FIGS. 11A-11D are the blood flow control device and loading sheath for inserting the blood flow control device.

FIG. 12 is a detailed view of a distal end of the blood flow control device anchor as described herein.

FIGS. 13A-13B show a situation in which blood may leak within a gap between the catheter and the anchor.

FIGS. 14-18B provide embodiments of solutions to filling or blocking the gap between the catheter and anchor.

FIGS. 19A-19C show examples of a handle of a flow control device.

### DETAILED DESCRIPTION

Described herein are catheter-based blood flow control devices that can be placed either in a blood vessel such as the SVC to decrease blood flow returning from the head and upper extremities or placed in the IVC in an infrarenal location in order to decrease blood flow returning from the lower extremities. The devices can restrict blood flowing in the antegrade direction, resulting in a higher pressure on the inflow (proximal) side and a lower pressure on the outflow side, distal to the device. Selectively articulable openings at or near a distal end and proximal end of an anchor provide increased control. The arrangement and articulation of a flow control element provides adjustment to one or more sections of the anchor to control a rate of blood flowing therethrough.

In some aspects described herein, an exemplary blood flow control device can be disposed in the SVC above the right atrium of the patient's heart. The device can comprise an expandable anchor supported by a catheter. The anchor can be configured to expand to engage the inside wall of the blood vessel (e.g., the SVC). As will be described below, the anchor can include a scaffold formed as a self-expandable stent covered by a blood impermeable covering to define a blood flow path extending from a proximal opening of the anchor to and through a distal opening of the anchor. A reduced diameter portion of the anchor creates at least a partial occlusion to blood flow through the device by reducing the area through which the blood is flowing.

In some aspects, the blood flow control device can be delivered to or placed within the IVC, e.g., just below the renal veins. As described herein, the device can include an expandable anchor supported by a catheter. The anchor is configured to expand to engage the inside wall of the blood vessel (e.g., the IVC). The anchor can include a scaffold formed as a self-expandable stent covered by a blood impermeable covering to define a blood flow path extending from proximal opening to and through a distal opening. A reduced diameter portion of the anchor creates at least a partial occlusion to blood flow through the device by reducing the area through which the blood is flowing.

The blood flow control devices described herein can include an expandable and compressible anchor that, in its expanded state, can accommodate a range of IVC or SVC diameters and seal against the IVC or SVC. In some embodiments, for example, the anchor can have an expanded diameter of 20-30 mm. The blood flow control devices described herein can further be compressible to a small enough diameter to be inserted via an introducer sheath placed in a peripheral vein, for instance inserted into the subclavian vein (for placement in the SVC location) or into the femoral vein (for placement in the IVC location).

The blood flow control devices described herein can have a non-thrombogenic surface on their inside diameter. In some embodiments, the flow occlusion devices can have minimal or no stent wires and/or no catheter shaft within the flow lumen in order to decrease the risk of thrombus forming on the device.

In some embodiments, the blood flow control devices described herein can be cylindrical. In other embodiments, the flow occlusion devices can have a narrowed location along their length in order to decrease the flow rate therethrough.

The blood flow control devices described herein can have a blood flow control element configured to vary the flow rate through the device, such as from fully open to partially or fully closed.

The blood flow control devices described herein can be configured to provide feedback to the physician. For example, the flow occlusion devices described herein can include pressure sensors supported by the catheter on or near the anchor and/or pressure-measuring lumens in the catheter communicating with ports distal and proximal to the device and leading to pressure sensors outside of the patient. As another example, the blood flow control devices described herein can include a flow rate sensor (e.g., within the narrowed location of the device or positioned distally and proximally to the device on the catheter). In some embodiments, the data from the pressure and/or flow sensors can be used by the physician to make adjustments to the blood flow control device to vary the flow rate or pressure differential as desired. In other embodiments, the data from the pressure and/or flow sensors can provide input to a controller, which can then automatically adjust the blood flow control device to vary the flow rate or pressure differential as desired. In some embodiments, other parameters may be used as the basis for adjusting the blood flow control device, such as right atrial pressure, pulmonary pressure, pulmonary capillary wedge pressure, urine output, and the like.

The blood flow control devices described herein can include an expandable and compressible anchor that, in its expanded state, can accommodate a range of IVC or SVC diameters and seal against the IVC or SVC. In some embodiments, for example, the anchor can have an expanded diameter of 20-30 mm. The blood flow control devices described herein can further be compressible to a small enough diameter to be inserted via an introducer sheath placed in a peripheral vein, for instance inserted into the subclavian vein or the jugular vein (for placement in the SVC location) or into the femoral vein (for placement in the IVC location). Additionally, physicians may desire to percutaneously insert the blood flow control devices superior to the renal veins by accessing the vasculature from a location such as the subclavian vein, jugular vein, etc. Depending on the access location, the orientation of the blood flow control device on the delivery catheter may need to be adjusted (e.g., for superior access, the proximal opening of the flow control device may be oriented with the distal end of the delivery catheter, while for inferior access, such as from the femoral vein, the distal opening of the flow control device is oriented with the distal end of the delivery catheter).

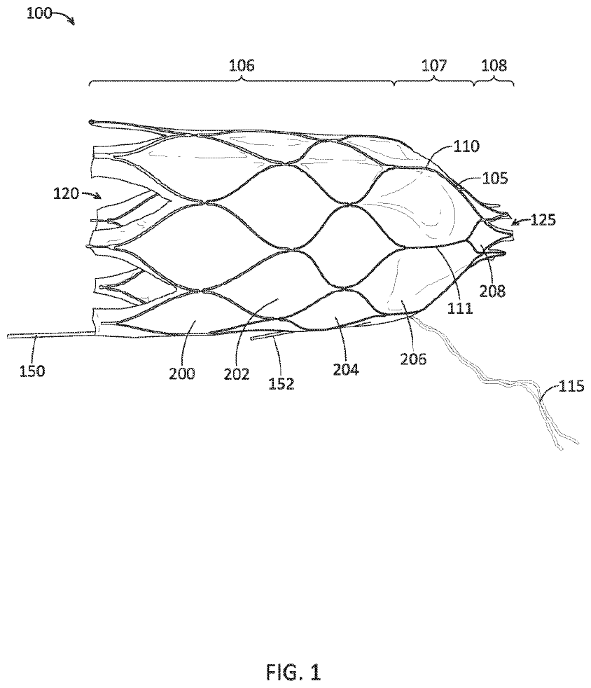

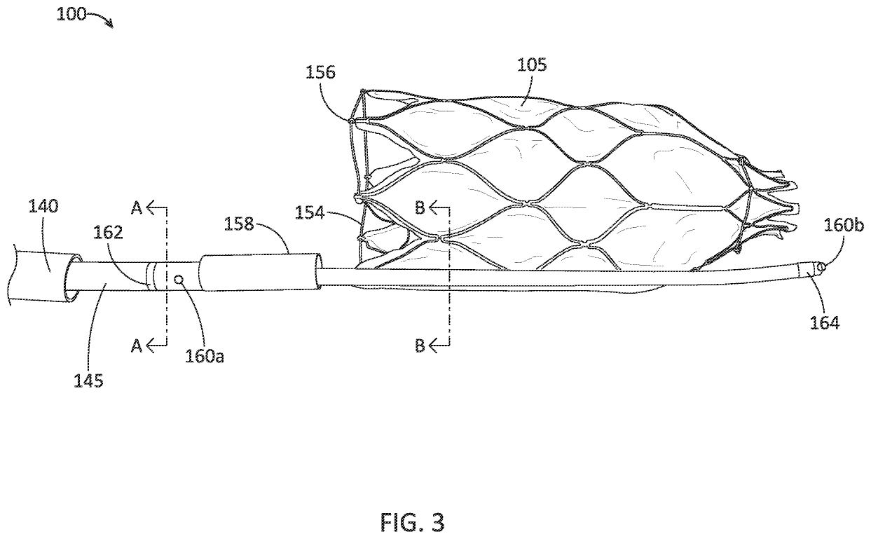

FIGS. 1-8B show an example of a blood flow control device 100. An anchor 105 supported by, and operably engaged with, a catheter 145 (shown in at least FIGS. 3, 4A-4B, 5A-5B, and 8A-8B) is expandable from a compressed delivery configuration toward an expanded configuration (as shown, e.g., in FIGS. 1-3) to engage an inside wall of a blood vessel so that nearly all of the blood flowing in the blood vessel is directed along a blood flow path through anchor 105. In some examples, blood may flow through the anchor. In some examples, there may be quantity of blood flow through the vessel between an exterior of the anchor and the interior of the vessel. In some examples, all or substantially all of the blood flowing through the vessel at the anchor may flow through an interior of an anchor.

The anchor 105 has one or more sections having an adjustable diameter. In the expanded configuration, a proximal section 106 extends distally from a proximal opening 120 and has a substantially cylindrical shape. A distal section 108 extends proximally from a distal opening 125 and also has a substantially cylindrical shape with a smaller diameter than the proximal section 106. A central section 107 tapers between the larger diameter proximal section 106 and the smaller diameter distal section 108. Sections 106, 107, and 108 and openings 120 and 125 are disposed on a common central axis.

In this example, the anchor 105 has a scaffold formed as a self-expandable stent 110 that can be covered on its inner and/or outer surface by a blood impermeable covering 130 to define a blood flow path extending from proximal opening 120 to and through a distal opening 125. Stent 110 may be formed, e.g., from an alloy, metal, or shape memory material such as nitinol, elgiloy alloy, steel (e.g., stainless steel), etc. In some examples, one or more cells in the stent may comprise a different segment length relative to one or more other cells in the array in an uncompressed state. In some examples, strut thickness, length, width, or combination thereof may be uniform throughout the stent. In some examples, one or more cells, portions, regions, segments, areas, etc. may have different or non-uniform strut thickness, length, width, geometry, or combination thereof. For example, some or all portions of the struts in the distal cell row may be narrowed to facilitate compression of the distal portion when cinching.

In the illustrated embodiment, stent 110 has five rows of cells 200, 202, 204, 206, and 208 defined by struts 111. Proximal cell rows 200, 202, and 204 have cells that are substantially the same size and shape. Cell row 206 extends over a distal portion of proximal section 106 through central section 107 into a proximal portion of section 108 and has cells that are larger than the cells of rows 200, 202, 204, and 208. The larger cells may provide increased flexibility for compression during retraction of the anchor into the sheath and/or for the stent's interaction with the flow control element, as discussed below.

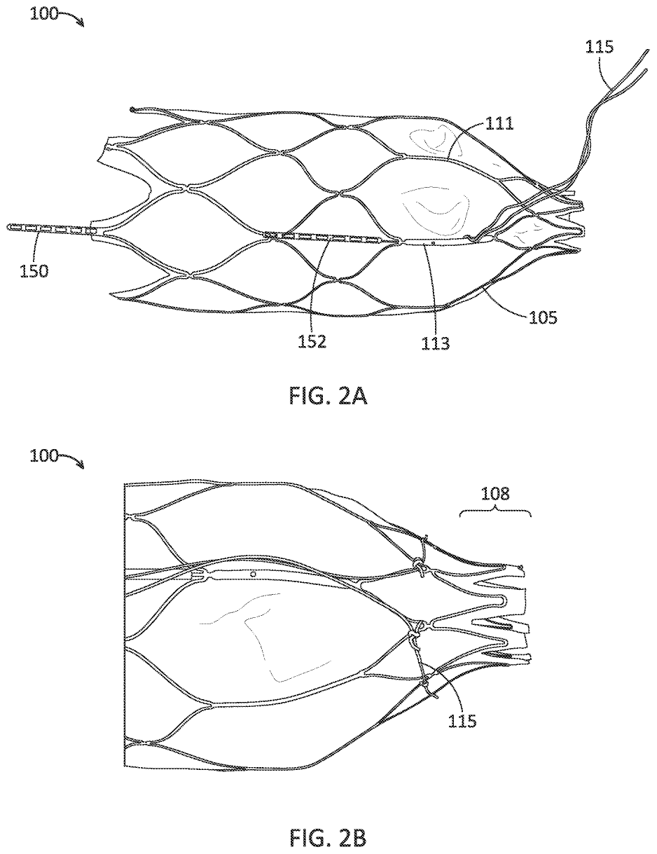

In some embodiments, referring to FIG. 2A, at least one of the struts 113 is stiffer (due, e.g., to being wider) than other struts 111 to form an anchor distal support adapted to resist movement of the distal portion of the anchor toward the catheter during actuation of the distal flow control element 115, as discussed below. In other embodiments, more than one strut can be wider or otherwise stronger to form the anchor distal support. In some examples, a size or geometry of one or more cells may comprise a proximal portion of the cell that is shorter in length than a distal portion of the cell. For example, one or more cells may be longitudinally asymmetrical and comprise distal struts that are longer than the proximal struts. In some examples, one or more cells may be longitudinally asymmetrical and comprise proximal struts that are longer than the distal struts. In some examples, one or more struts of a cell may me thicker, thinner, or a combination thereof compared to other struts of the same cell or other struts of another cell.

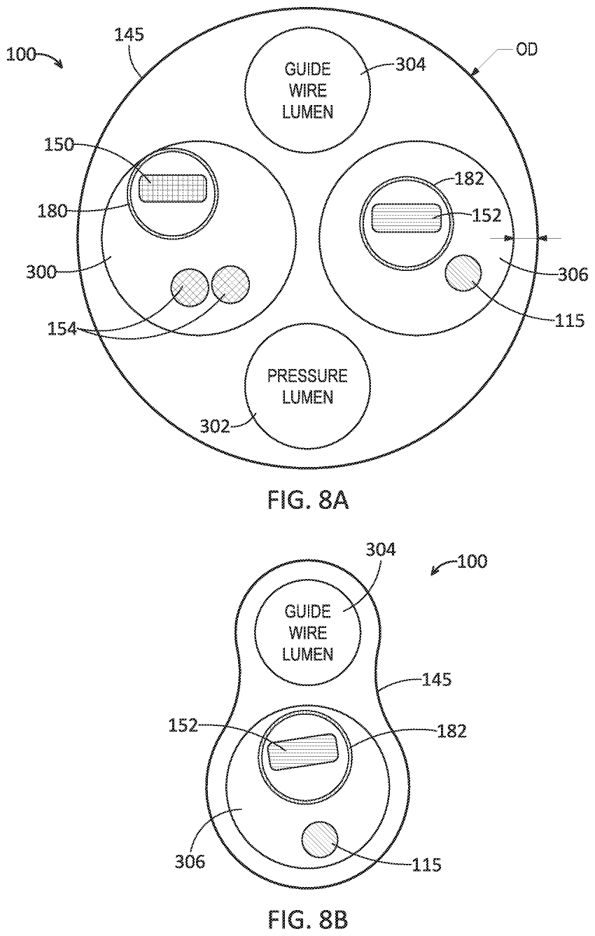

Referring to FIG. 3, catheter 145 supports anchor 105 outside of the blood flow path through anchor 105. Proximal and distal catheter attachment elements 150 and 152 may be formed on, or attached to, the proximal and distal ends of stent 110. The orientation of the anchor 105 on the catheter 145 as shown in FIG. 3, with the distal opening of the anchor oriented with the distal end of the catheter, is suitable for access to a target vessel location from an inferior location (e.g., from a femoral vein access point). As described above, if access is desired from a superior location to the target vessel site (e.g., from a subclavian or jugular vein), then the anchor can be flipped on the catheter such that the proximal opening is oriented with the distal end of the catheter. In some embodiments, one or both of the catheter attachment elements may be slidingly disposed in a lumen of catheter 145 so that one or both ends of the stent can move with respect to the catheter as the stent expands or is compressed. Instead of sliding in a lumen of the catheter, one of the attachment elements may be fixed to the catheter by adhesive or by polymer melt bonding in a lumen of the catheter or on an outside surface of the catheter. For example, distal catheter attachment element 152 may be slidingly disposed in a lumen 306 of catheter 145, as shown in FIGS. 8A and 8B, and proximal catheter attachment element 150 may be fixedly attached to the interior of another lumen 300 of catheter 145, as shown in FIG. 8A. Alternatively, proximal catheter attachment element 150 may be slidingly disposed in a catheter lumen, and the distal catheter attachment element 152 may be either fixedly or slidingly disposed in a catheter lumen. In yet another alternative embodiment, the proximal and/or distal attachment element may be bonded to a tube, and the tube may be slidingly disposed in a lumen of the catheter, or the tube may be bonded to the catheter lumen.

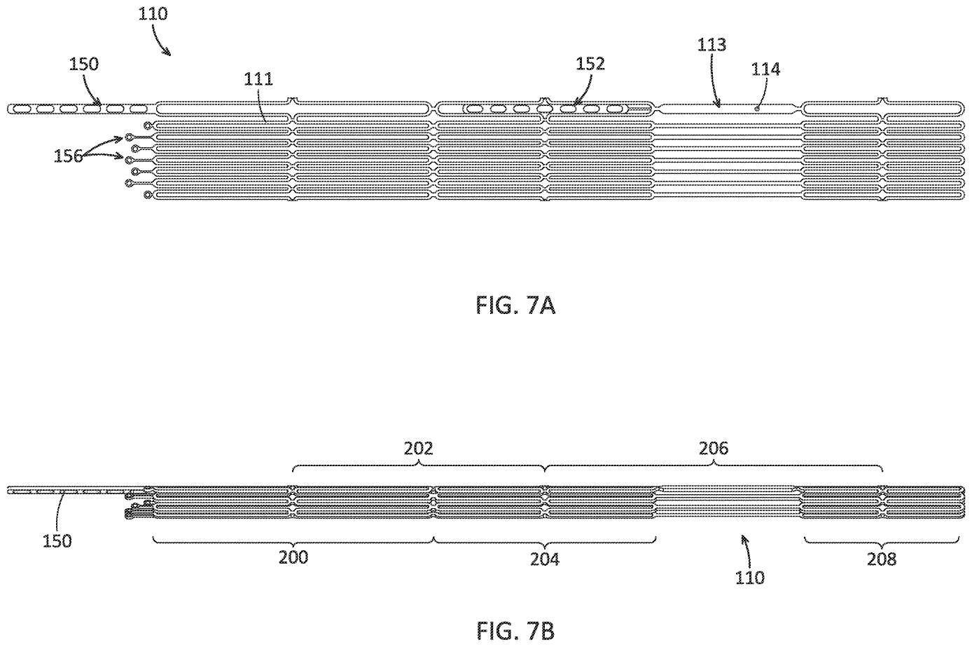

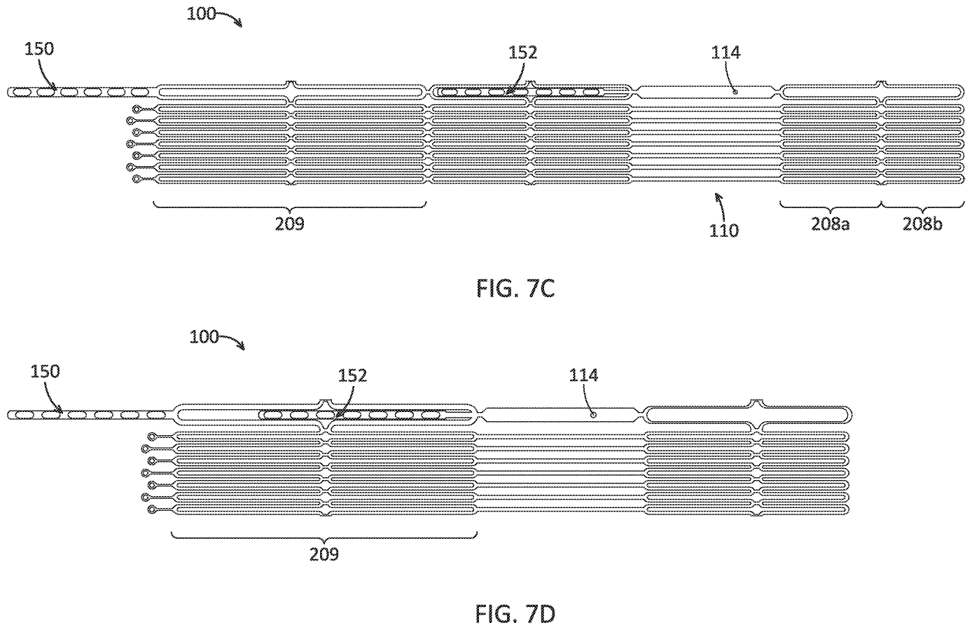

Referring to FIGS. 2A-2B, a flow control element 115 (such as a suture thread) forms a cinch line that encircles and engages the anchor 105 within section 107 or section 108. Flow control element 115 may extend around the outer surface of anchor 105, as shown, or may extend around an inner surface of anchor 105, or a combination thereof. Flow control element 115 may be used to control a rate of blood flowing through the anchor 105 from a proximal opening 120 to the distal opening 125. Flow control element 115 may extend out of a lumen 306 of catheter 145 (shown in FIGS. 8A-8B) and through a hole 114 in stiff strut 113 to anchor 105 (shown in FIGS. 7A, 7C, and 7D). Alternatively, flow control element 115 may extend through a loop formed in, or attached to, stiff strut 113 before encircling the anchor.

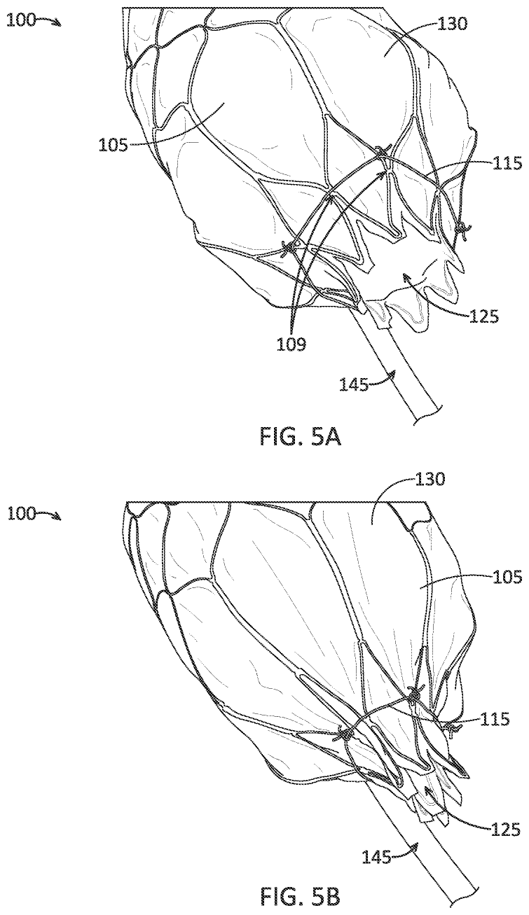

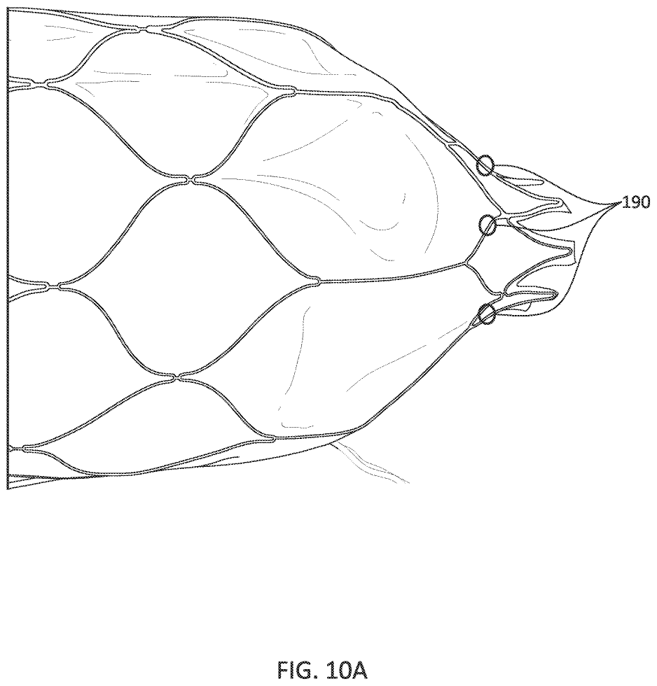

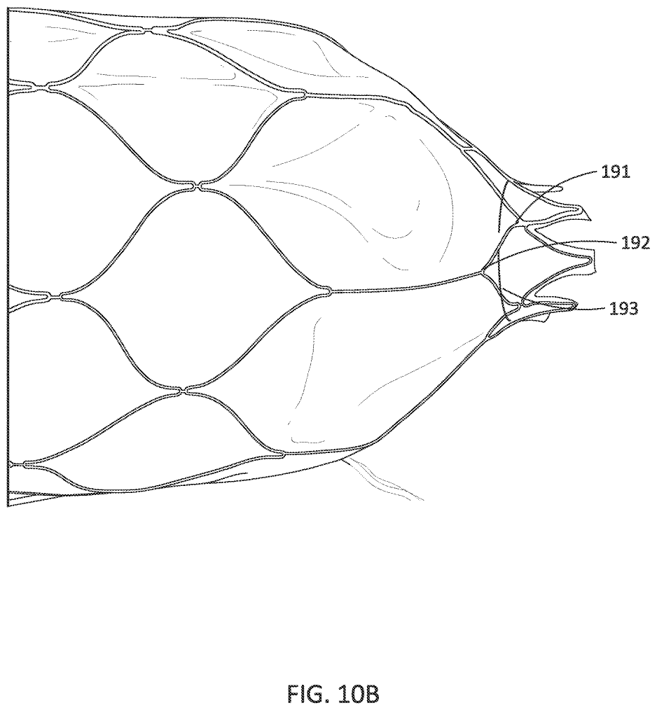

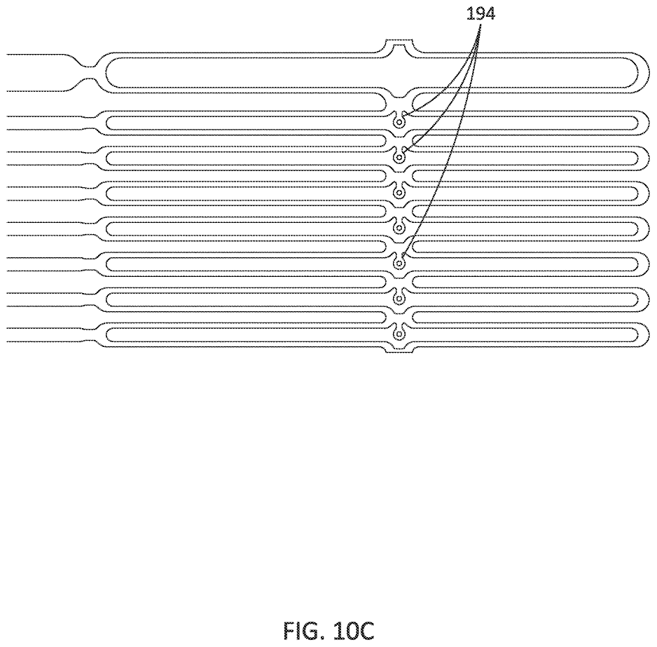

As shown, for example in FIGS. 5A and 5B, flow control element 115 may be attached to anchor 105 by suture loops 109 or other attachment features (e.g., stainless steel rings) at the distal end of stent cell row 206 in a central region of cell row 208. Suture loops may extend through the anchor covering 130 to extend around the stent struts. Alternatively, if the flow control element 115 extends around the inner surface of anchor 105, the supporting loops may be disposed on the inner surface of stent 110. In yet another alternative, the cinching line may weave through and around the stent struts without employing support loops, as illustrated in FIG. 10B.

One or both proximal ends of flow control element 115 may extend proximally through catheter 145 to an actuator (not shown), as described in U.S. Pat. No. 11,638,585, which is incorporated herein by reference in its entirety. When actuated, flow control element 115 cinches the distal section 108 of anchor 105 to reduce its diameter, thereby increasing the occlusion or restriction of blood flowing therethrough, while still maintaining its substantially cylindrical shape. The stiff strut 113 helps resist downward movement of anchor 105 toward catheter 145 when flow control element 115 is pulled and cinched. The cylindrical geometry of the distal section 108 in compressed and uncompressed configurations can reduce the turbulence of blood flowing therethrough. For example, the uniform diameter of the distal section may provide a laminar flow of blood passing therethrough while reducing turbulence after the blood leaves the anchor 105 through the distal opening 125.

In some examples, the distal section 108 may have a geometry adjustable by the flow control element. For example, as the flow control element is engaged, the distal section 108 may have a geometry that changes. In some examples, the distal section 108 may have a generally cylindrical geometry with a diameter less than one or more other sections of the anchor 105 (e.g., sections 106 and 107). In some examples, the flow control element may cinch and adjust the geometry of the distal section from a conical geometry, to a generally cylindrical geometry, and to a flared geometry, or a combination thereof. In some examples, the distal section 108 may comprise a flared distal opening where the proximal side of distal section 108 has a diameter that is less than the proximal sections (e.g., 106 and 107) and less than the distal perimeter. As illustrated in FIG. 2B, the distal end of section 108 is flared where the distal ends of the distal cells are flared open as the flow control element engages the distal end.

Referring to FIG. 3, the catheter 145 may extend through a delivery catheter or sheath 140. Sheath 140 may be advanced through the patient's vasculature to the intended implantation site of the blood flow control device 100 in the IVC or SVC. Once at the site, catheter 145 may be advanced, or sheath 140 may be retracted, to allow anchor 105 to emerge from sheath 140 from a compressed delivery configuration toward an expanded configuration to contact the blood vessel wall, thereby directing all blood to flow into proximal opening 120, through the interior blood flow path defined by anchor 105, and out distal opening 125. In the illustrated embodiment, anchor 105 is self-expandable. In some embodiments, a short loading sheath may be used to compress the anchor 105 prior to loading the anchor and catheter 145 into sheath 140.

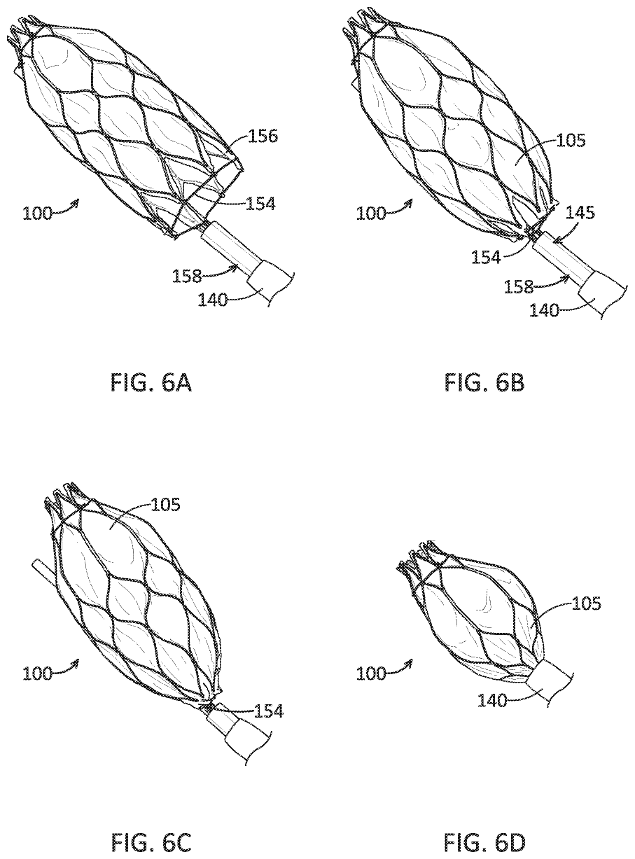

Also shown in FIG. 3 is a proximal flow control element (or cinch line) 154 extending through proximal loops 156 formed in, or attached to, stent 110. Proximal flow control element 154 extends proximally to an actuator that may be used to tighten proximal flow control element 154, thereby pulling loops 156 inward to compress the proximal end of stent 110 during retraction of anchor 100 into the sheath 140, as shown in FIGS. 6A-6D. An optional ramp 158 may be provided on a top side of catheter 145 proximal to anchor 105 to engage with and move the distal end of sheath 140 upward to align the distal opening of sheath 140 with the proximal end of the anchor 105 during retraction of anchor 105 into sheath 140.

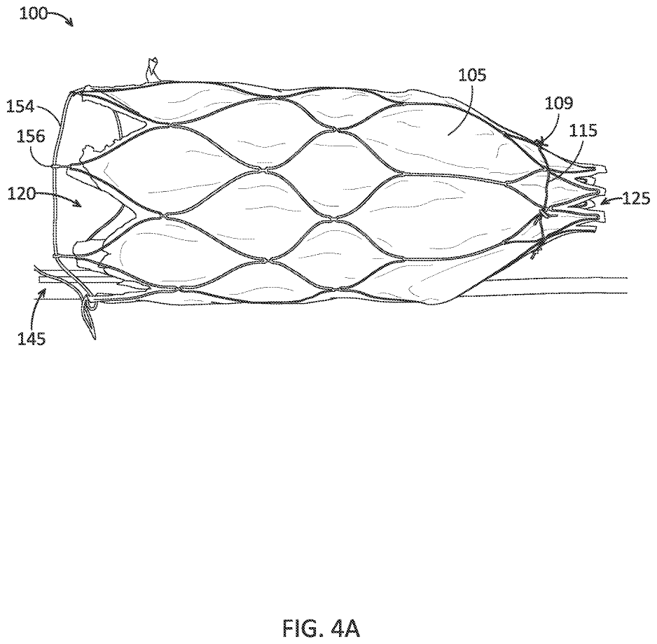

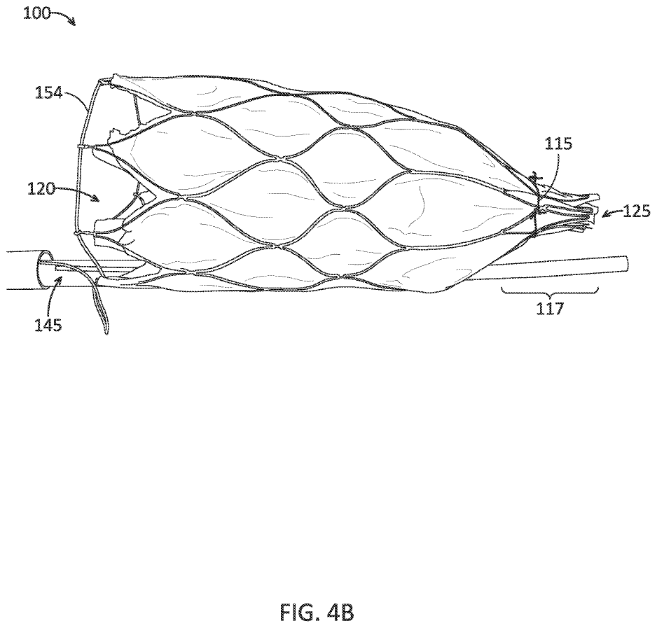

Similar to FIG. 2B, FIG. 4B illustrates another examples of an outward flare geometry of the distal section of the anchor. The characteristics (e.g., attributes) of one of more struts of a cell may facilitate the adjustable geometry of the distal section. For example, the distal cell row 117 may comprise cells having distal struts that are thicker than the proximal struts. Accordingly, when the flow control element (e.g., cinch line) 115 is engaged around the distal cell row 117, and particularly around the thinner proximal struts of distal cell row 117, the distal section decreases in diameter and can flare outward or open to have a larger diameter than the diameter of the distal section engaged by the flow control element.

The thicker distal struts of the distal cell row 117 may be less flexible than the proximal struts and facilitate the outward flare as the flow control element is engaged.

FIGS. 7A-D show examples of stent scaffold of a blood flow control device. In some examples, cell rows (e.g., 200, 202, 204, 206, 208, etc.) comprise struts having characteristics (e.g., stiffness, length, thickness, geometry, etc.) that facilitate geometric adjustment of one or more sections of the blood flow control device to adjust a flow rate of blood flowing therethrough. A cinch line can engage the stent 110 routed through one or more holes 114 and attachment elements to retain the cinch line in a position relative to one or more cell rows. In some examples, attachment elements may be locatable on the stent to maintain a route or position of the cinch line.

Referring to FIG. 7C, the distal cell row 208 is shown having a proximal half 208a and a distal half 208b of each cell within the row. Here, the proximal half 208a is lengthened relative to the distal half 208b. The cinch line may be maintained around the cell row proximal half 208a and when the cinch line is engaged, forces supplied by the cinch line on the proximal half 208a can compress or reduce the diameter of the distal cell row forming the distal section of the blood flow control device. In some examples, struts of the stent may be thicker or narrower (e.g., thinner) relative to other struts to facilitate the change in geometry of the blood flow control device when the cinch line cinches the device. For example, a thinner strut may change a shape when the cinch line is cinched to provide increased change in the cell and/or section geometry. As described herein, the distal section of a blood flow control device may be generally cylindrical as supported by the strut characteristics and differential response to cinching by the cinch line.

An anchor may comprise a stent scaffold with any number of cell rows. FIG. 7D illustrates an example of a stent scaffolding having fewer cells rows than examples illustrated in FIGS. 7A-7C. The distal attachment element 152 may be positioned according to the number of cell rows of the stent scaffolding. The length of the stent scaffolding of the anchor may comprise one or more rows of cells. In some examples, a stent scaffold may comprise one row of cells, two rows of cells, three rows of cells, four rows of cells, five rows of cells, six rows of cells, or more.

In some examples, the blood flow control device of this invention may be used to regulate (e.g., limit) the return rate of blood to the heart, thereby ultimately lowering blood pressure within a blood vessel, e.g., in the SVC or in the IVC. Lowering blood pressure in the SVC or in the IVC may also lower the cardiac filling pressure and preload on the right side of the patient's heart and which may be beneficial in treating heart failure. In some examples, the blood flow control device of this invention may be used to regulate (e.g., limit) the return rate of blood to the heart, thereby lowering the cardiac filling pressure and preload on the right side of the patient's heart and which may be beneficial in treating heart failure. In addition, placement of the device adjacent the renal veins may decrease the blood pressure at the outlet of the renal veins and thereby increase blood flow through the kidneys. One or more pressure sensors may be used to determine the pressure differential across the device.

In the embodiment shown in FIGS. 1-8B, and particularly FIG. 3, a port 160a on catheter 145 proximal to anchor 105 may communicate blood pressure at that point via a fluid column in pressure lumen 302 (shown in FIG. 8A) in the catheter to a first pressure sensor (not shown) on the proximal end of catheter 145. Likewise, a port 160b at the distal end of catheter 145 may communicate via a fluid column in a separate lumen 304, such as the guidewire lumen (shown in FIGS. 8A and 8B) in the catheter to a second pressure sensor (not shown) on the proximal end of catheter 145. Lumen 304 may also be used as a guidewire lumen for delivery of blood flow control device 100 to the desired site in the blood vessel.

Alternatively, instead of communicating blood pressure via lumens 302 and 304 to remote pressure sensors at the proximal end of catheter 145, MEMS or other pressure sensors may be mounted on the catheter just distal to and just proximal to anchor 105, and wires may extend from the pressure sensors through the catheter lumens to a processor at the proximal end of catheter 145.

Radiopaque markers 162 and 164 may be provided for fluoroscopic viewing of the delivery and implantation of device 100, and may also provide an indication of the location of the proximal and distal pressure sensors to ensure proper placement of the device and pressure sensors before deployment.

FIG. 8A is a cross-sectional view of catheter 145 taken along line A-A of FIG. 3, and FIG. 8B is a cross-sectional view of catheter 145 taken along line B-B of FIG. 3. As shown in FIG. 8A, catheter 145 has four lumens at point A-A proximal to anchor 105.

Proximal anchor attachment element 150 is bonded to a tube 180, and tube 180 is bonded to the interior of catheter lumen 300. The proximally-extending ends of the proximal flow control element 154 are also disposed in lumen 300. A pressure lumen 302 communicates pressure port 160a with a pressure sensor at the proximal end of device 100.

Lumens 300 and 302 end proximal to the distal end of catheter 145. As shown in FIG. 8B, a guidewire lumen 304 extends beyond the ends of lumens 300 and 302 to an opening or port 160b at the distal end of catheter 145. Guidewire lumen 304 also may serve as a pressure lumen to communicate blood pressure distal to device 100 to a pressure sensor at the proximal end of device 100. A second lumen 306 extends beyond the ends of lumens 300 and 302. Distal anchor attachment element 152 is bonded to a tube 182 that is slidable within lumen 306 so that the distal end of anchor 105 can move distally with respect to catheter 145 during compression and proximally during expansion while the proximal end of anchor 105 remains stationary. The cinch line(s) of flow control element 115 also extend proximally through lumen 306.

The blood flow control devices described herein can include an expandable and compressible anchor that, in its expanded state, can accommodate a range of IVC or SVC diameters and seal against the IVC or SVC. In some embodiments, for example, the anchor can have an expanded diameter of 20-30 mm. The blood flow control devices described herein can further be compressible to a small enough diameter to be inserted via an introducer sheath placed in a peripheral vein, for instance inserted into the subclavian vein (for placement in the SVC location) or into the femoral vein (for placement in the IVC location).

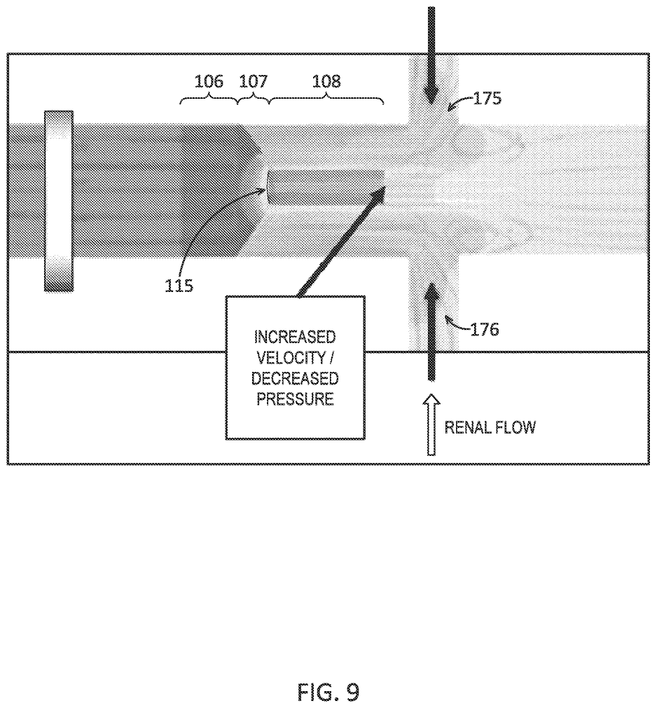

In FIG. 9, an example of a blood flow control device is shown placed within an IVC. The distal section 108 is illustrated with a smaller diameter than the proximal section 106 with section 107 tapered therebetween. Cinching distal flow control element 115 supports the change in geometry and diameter of distal section 108. Adjusting the flow control element 115 can control the pressure and velocity of blood flow through the device and proximal vessels. For example, the placement and configuration of the blood flow control device illustrated in FIG. 9 can provide a venturi effect to increase velocity of blood flowing from the distal section 108 and decrease pressure of the blood flow. Flow rate from the renal veins 175 and 176 can be increased based on the adjustment of blood flow through the device. In some examples, a venturi effect when the anchor is placed near the renal veins can provide a lower pressure region and can draw in more blood from the renal veins associated with a higher velocity in the flow exiting the distal end of the anchor.

In some examples, a cinch line may engage the blood flow control device as facilitated by suture loops locatable around the stent scaffold. In addition to or alternatively, one or more attached features may support the cinch line routing and engagement with blood flow control device. FIGS. 10A to 10C illustrate examples of attachment features. As described herein, suture loops may be locatable on the stent and can provide a route or path for a cinch line to pass therethrough. In some examples, attachment features can include rings 190 that may be attached, or incorporated into the stent where the cinch line is to be routed. These rings 190 can provide rigid attachment elements or points for the cinch line and support maintaining the cinch line in position around the stent. For example, rings 190 may be crimped around the struts before ePTFE coating. In some examples, attachment features can include contact areas of adjacent cells. For example, a cinch line 193 may be routed through cells as illustrated in FIG. 10B where a cinch line may be routed through the proximal end 192 of a first cell and a distal end 191 of an adjacent cell. The routing of a cinch line can provide alternative engagement of the cinch line with the stent where the cinch line is partially on the inside diameter of the stent and partially on the outside diameter of the stent. In some examples, attachment features can include holes or eyelets 194 incorporated into the stent pattern itself. These eyelets 194 may be bent outward when heat-setting the nitinol. In some examples, a channel or other attachment feature may be incorporated into the stent pattern that can be configured to retain the cinch line in position relative to a cell row.

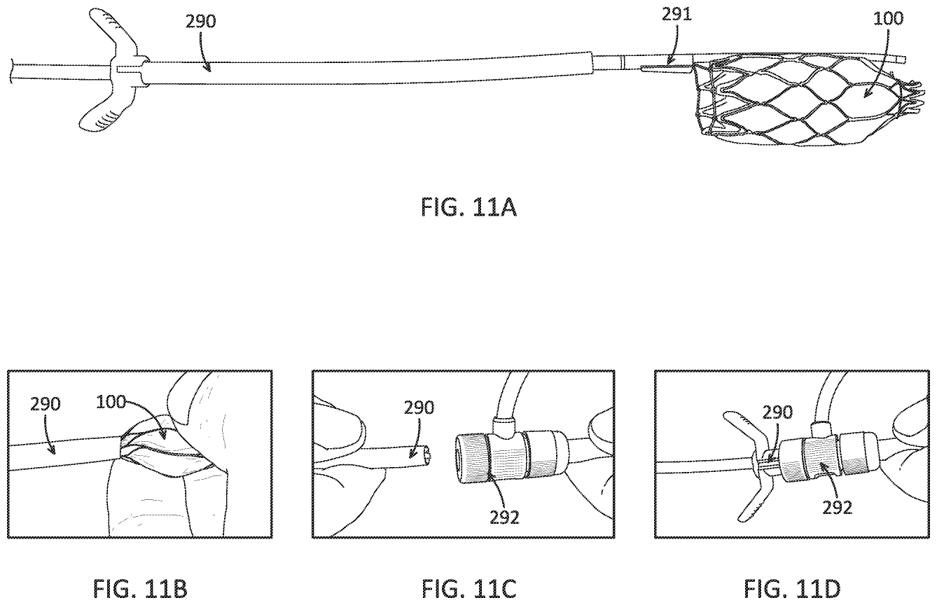

FIGS. 11A-11D illustrates an examples of loading a blood flow control device into a vascular sheath. FIG. 11A shows the sheath 290 over the catheter 291 and the blood flow control device 100 in an expanded state. FIGS. 11B-11D illustrate examples of steps related to loading the blood flow control device into the sheath 290 and the sheath 290 loaded into a vascular sheath through a hub 292. FIG. 11B shows the blood flow control device 100 being compressed as it enters the sheath 290. Once loaded, the sheath 290 can be introduced into a vascular sheath via a hub 292 and the blood flow control device can be advanced through the vascular sheath as illustrated in FIG. 11D.

The blood flow control devices described herein can have a non-thrombogenic surface on their inside diameter. In some embodiments, the flow occlusion devices can have minimal or no stent wires and/or no catheter shaft within the flow lumen in order to decrease the risk of thrombus forming on the device.

The blood flow control devices described herein can have a blood flow control element configured to vary the flow rate through the device, such as from fully open to partially or fully closed.

A handle disposed at the proximal end of catheter 145 may support actuators for actuating the flow control element 115 and/or the proximal flow control element 154. Examples of such handles may be found in US Pat. Publ. No. 2022/0287831.

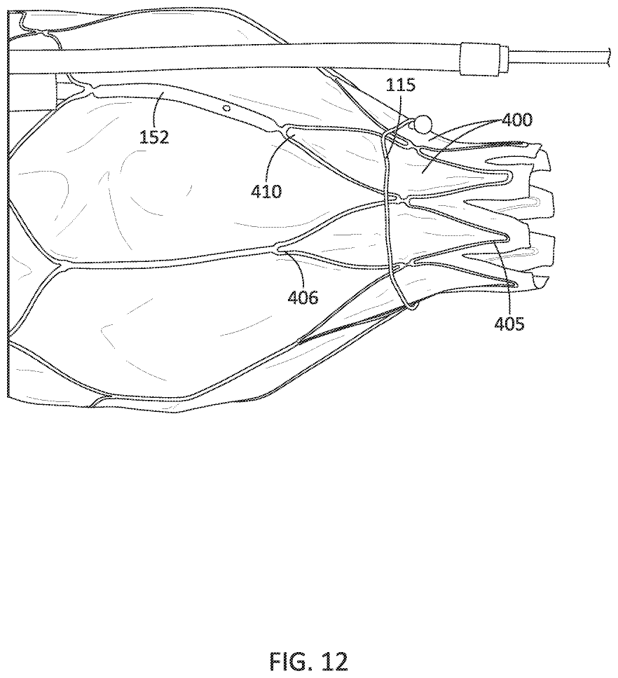

In some examples, the blood flow control devices described herein may comprise cells having a proximal angle 406 or distal angle 405 configured to facilitate operation of the device. FIG. 12 illustrates an example of distal cell configuration for a blood flow control device anchor with details of the distal cells 400 each having a proximal angle 406 and distal angle 405. In an expanded state, the distal angle 405 and proximal angle 406 of a cell may be at some angle between 1 degrees and 90 degrees or more. In some examples, a smaller angle (e.g., 405, 406, 410, etc.) may provide stability and structural integrity to the scaffolding as the flow control element 115 is engaged. In some examples, a larger angle between the cell struts may provide increased flexibility to the cell. Referring specifically to the catheter attachment element 152, at a distal end of the attachment element, a proximal cell angle 410 may comprise strut attributes, different than other struts of the stent. For example, angle 410 may be smaller than the proximal angle of other cells to facilitate the stent structure, prevent bending of the distal section of the anchor (e.g., stent), modulate changes to the distal section geometry when the flow control element is engaged, etc., or a combination thereof. In some examples, angle 410 may be 45 degrees or less. In some examples, angle 410 may be between 20 degrees and 40 degrees in an expanded state. In some examples, angle 410 may be less than 45 degrees in an expanded state. In some examples, angle 410 may be 45 degrees in an expanded state. In some examples, angle 410 may be greater than 45 degrees in an expanded state. In some examples, the remaining cells of the distal row (e.g., the row comprising cells with angle 410) may comprise a proximal angle greater than angle 410 distal to the attachment element 152.

Similarly, at a proximal section of an anchor (e.g., the stent scaffolding), proximal attachment element (e.g., element 150 illustrated in FIGS. 7A-7D) may be coupled to a cell at a distal end of the proximal attachment element and the proximal angle of such cell may be smaller than the proximal angle of other cells to facilitate the stent structure, prevent bending of the proximal section of the anchor (e.g., stent scaffolding), modulate changes to the proximal section geometry when the flow control element is engaged, when the anchor is retracted, etc., or a combination thereof. In some examples, the angle of the cell proximal to the distal end of the proximal attachment element may be 45 degrees or less in an expanded state, between 20 degrees and 40 degrees in an expanded state, less than 45 degrees in an expanded state, 45 degrees in an expanded state.

In some examples and in addition to the smaller angle of the cells proximal to the distal ends of each catheter attachment element, a width of associated struts may be larger (e.g., thicker) to provide additional structural integrity of the stent scaffolding, when the flow control element in engaged. For example, strut widths may be between 0.05 millimeters (mm) and 1 mm. In some examples, one or more strut widths may be between 0.10 mm and 0.40 mm In some examples, one or more strut widths may be between 0.20 mm and 0.35 mm (e.g., 20 m, 0.21 mm, 0.22 mm, 0.23 mm, 0.24 mm, 0.25 mm, 0.26 mm, 0.27 mm, 0.28 mm, 0.29 mm, 0.30 mm, 0.31 mm, 0.32 mm, 0.33 mm, 0.34 mm, etc.) In some examples, any strut width may be 1 mm, 0.12 mm, 0.13 mm, 0.14 mm, 0.15 mm, or more or less or any mm therebetween.

The blood flow control devices described herein can be configured to provide feedback to the physician. For example, the flow occlusion devices described herein can include pressure sensors supported by the catheter on the anchor, or on the catheter proximal and distal to the anchor, and/or pressure-measuring lumens in the catheter communicating with ports distal and proximal to the device and leading to pressure sensors outside of the patient. As another example, the blood flow control devices described herein can include a flow rate sensor (e.g., within the narrowed location of the device or positioned distally and proximally to the device on the catheter). In some embodiments, the data from the pressure and/or flow sensors can be used by the physician to make adjustments to the blood flow control device to vary the flow rate or pressure differential as desired. In other embodiments, the data from the pressure and/or flow sensors can provide input to a controller, which can then automatically adjust the blood flow control device to vary the flow rate or pressure differential as desired. In some embodiments, other parameters may be used as the basis for adjusting the blood flow control device, such as right atrial pressure, central veinous pressure, pulmonary artery pressure, pulmonary capillary wedge pressure, urine output, and the like.

The blood flow control devices described herein can be used in the SVC or IVC temporarily (e.g., for 8-72 hours) to decrease cardiac filling pressures and preload on the right heart. For example, the flow occlusion devices described herein can be placed in an infrarenal location of the IVC, which may advantageously additionally decrease the renal vein pressure, thereby increasing diuretic effectiveness. The flow occlusion devices described herein can be used to maintain a desired pressure differential thereacross. Advantageously, the flow occlusion devices described herein can achieve variable occlusion, enabling the user (e.g., physician) to adjust the occlusion as desired.

The blood flow control devices described herein can be used in the SVC or IVC for long term treatment with the anchor being deployed and left in the vessel (e.g., for more than 8-72 hours) to decrease cardiac filling pressures and preload on the right heart. For example, the flow occlusion devices described herein can be placed in an infrarenal location of the IVC, which may advantageously additionally decrease the renal vein pressure, thereby increasing diuretic effectiveness. The flow occlusion devices described herein can be used to maintain a desired pressure differential thereacross. Advantageously, the flow occlusion devices described herein can achieve variable occlusion, enabling the user (e.g., physician) to adjust the occlusion as desired.

Any or all of the blood flow control devices described above may have anchors that self-expand to 28 mm diameter with sufficient outward expansion force, and the device may be compressed to a size less than 16 F. The adjustable blood flow control devices described above may be controlled to restrict the blood flow area from a fully open configuration of, e.g., 9 14 mm diameter, to a fully closed configuration. The devices may have a length of 4 cm. The catheter may have a built-in loading sheath (e.g., 16 F sheath) for introduction into a 16Fr vascular sheath (e.g., a 18 F vascular sheath).

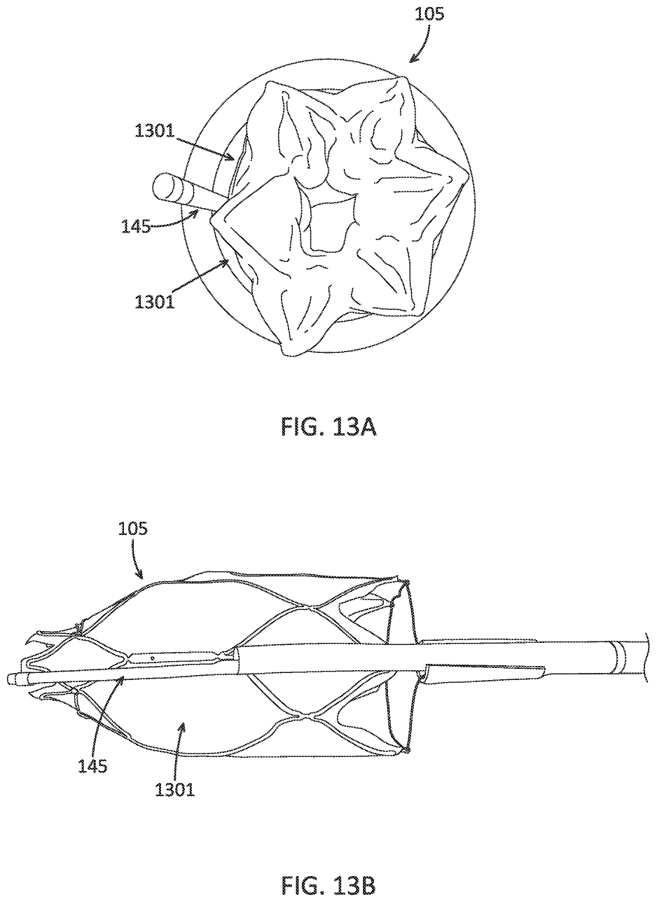

Referring now to FIGS. 13A-13B, in some instances leakage may occur along the catheter shaft which sits outside of the flow occlusion device and between the anchor and the vessel wall. In particular, leakage may occur within gaps 1301 formed between or along the catheter 145 and the anchor 105. This can result in inaccuracies in the measurement of pressure drops across the device.

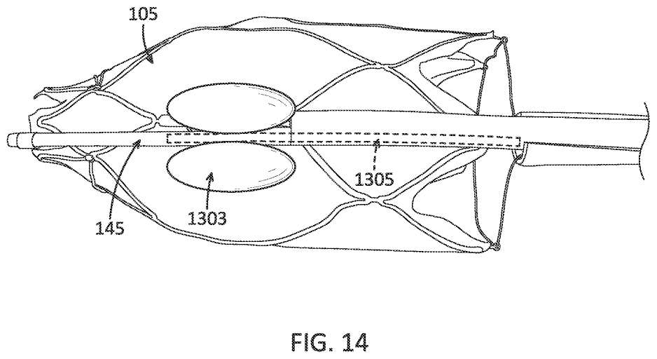

In one embodiment, referring to FIG. 14, active inflatable balloons or expandable elements 1303 can be inflated or expanded to fill or seal the gaps 1301 between the catheter 145 and the anchor 105. The inflatable balloons or expandable elements can be disposed along a portion of the catheter and the anchor near the gaps. In some embodiments, the catheter 145 can include another lumen 1305 fluidly coupled to the expandable elements to facilitate inflation or expansion. For example, saline, air, or another fluid may be delivered via lumen 1305 in the catheter 145 to cause the inflatable balloons or expandable elements to expand into the gaps to fill the gaps 1301. The expandable elements can comprise, for example, a soft conformable material configured to expand into the gaps 1301 without damaging the vessel walls. In some embodiments, the expandable elements can comprise a material different than the blood impermeable material of the anchor device. In other aspects, the material of the expandable elements can be the same as the blood impermeable material of the anchor device.

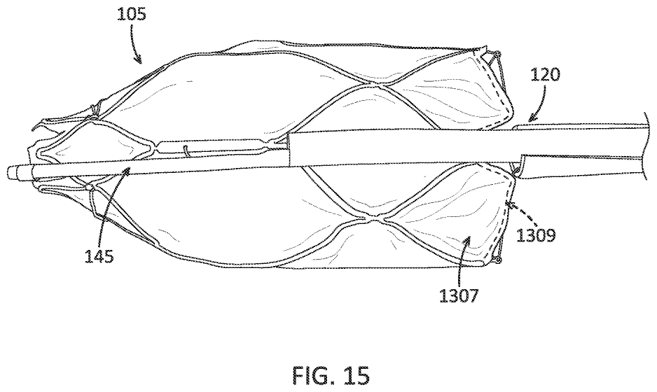

In another embodiment, referring to FIG. 15, additional material or anchor covering 1307 can be added between apices of the anchor 105, at the proximal (inflow) opening of the anchor. The additional material or anchor covering can include excess material in the space within the anchor defined by the apices of the anchor frame. For example, the surface area of the additional material between the apices may be greater than the area defined by the struts and apices of the anchor in that particular cell. When fluid or blood flows through the anchor and pressurizes the anchor, the fluid flow can cause the additional material or anchor covering 1309 to flare or expand outwards to a larger diameter than the rest of the anchor, causing the additional material to expand into the gaps 1301 to prevent leakage between the catheter and the anchor. In some embodiments, additional supports 1309 can be added between the apices, to provide additional support for the material 1309. In some aspects, the supports can comprise shape memory material such as nitinol that is pre-biased to expand outward towards the vessel wall to cause the material 1307 to expand into the gap.

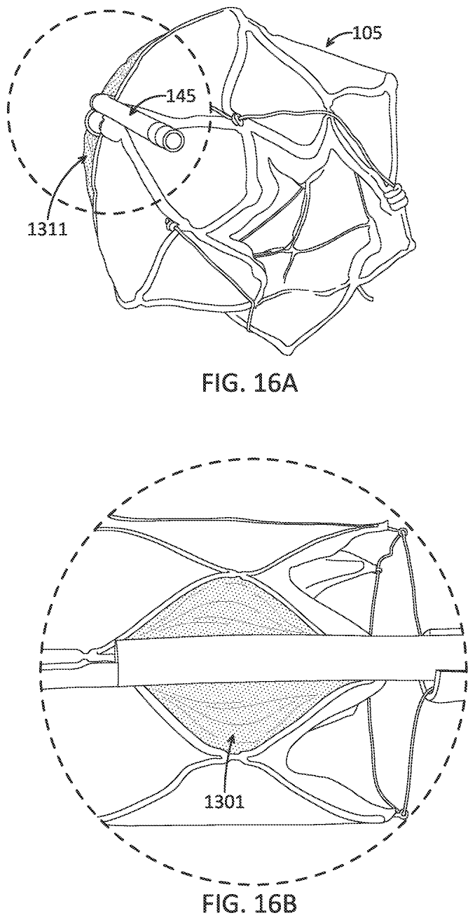

Referring to FIGS. 16A-16B, in another embodiment, the anchor can have additional material 1311 within the struts of one cell that spans or crosses the catheter 145.

Similar to the embodiment above, the additional material can comprise expandable elements configured to be expanded to move or expand outwards towards the catheter 145 and gaps 1301 when blood is flowing through the anchor 105.

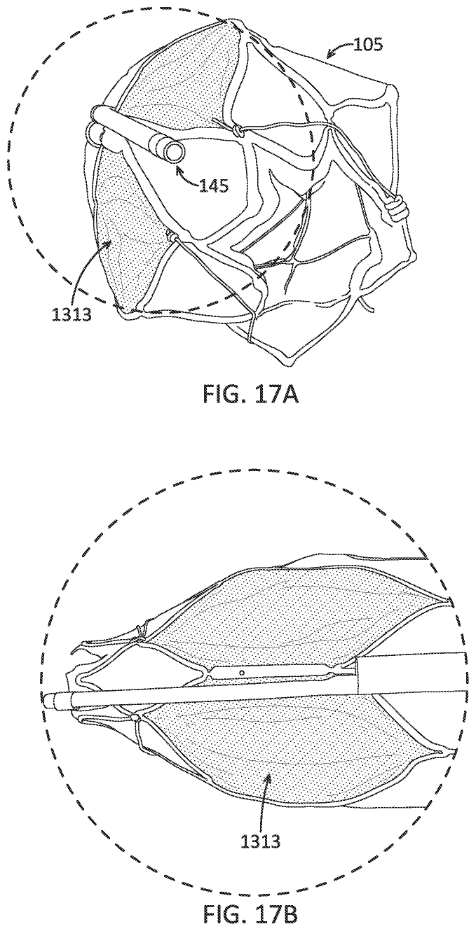

Referring to FIGS. 17A-17B, in another embodiment, the anchor can have additional material 1313 within the struts of two cells on either side of the catheter 145. Similar to the embodiment above, the additional material can be expanded to move or expand outwards towards the catheter 145 and gaps 1301 when blood is flowing through the anchor 105.

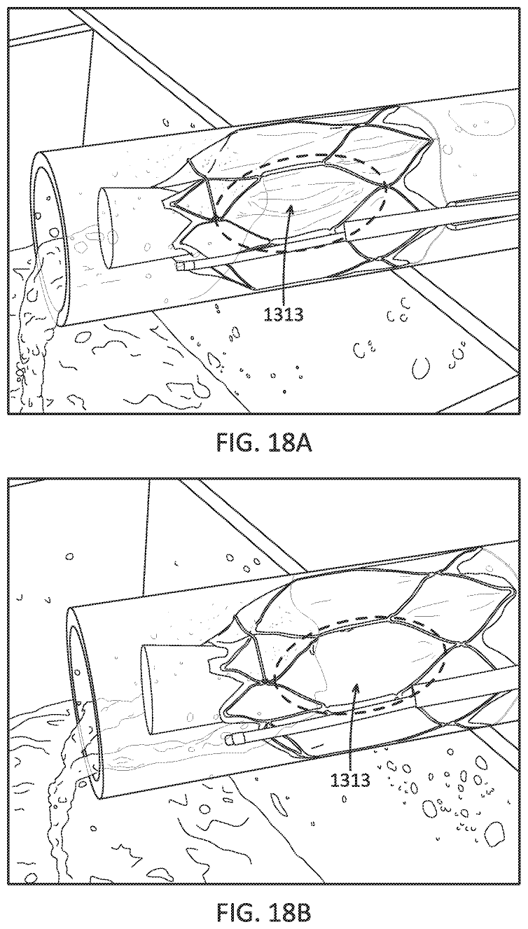

FIGS. 18A-18B show the additional material 1313 of FIGS. 17A-17B. In FIG. 18A, no blood or fluid is flowing through the anchor, so the excess material is not pressurized. In FIG. 18B, however, the additional material is pressurized by blood flowing through the anchor, causing it to expand outwards towards the vessel wall to fill the gaps formed between the anchor and the catheter.

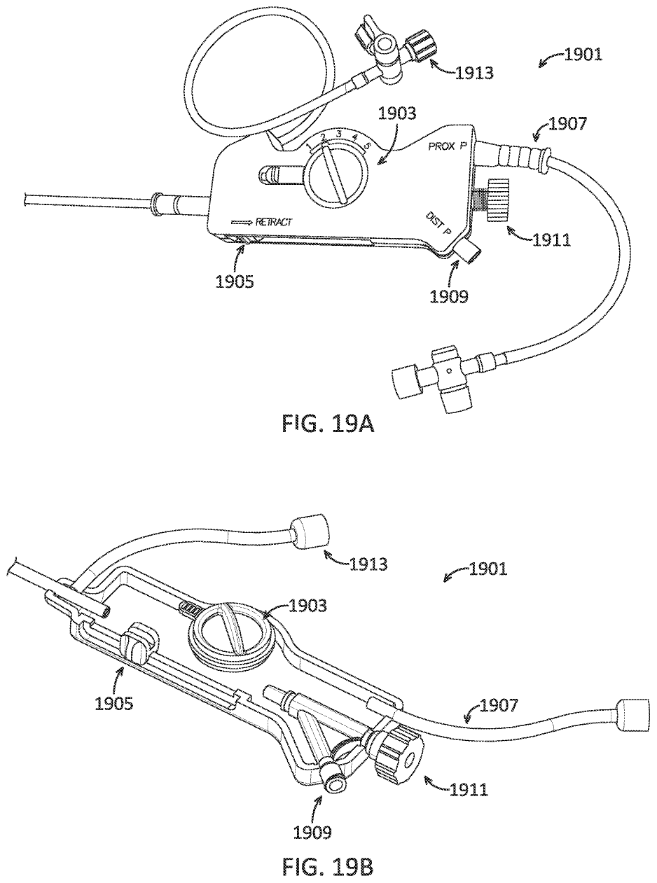

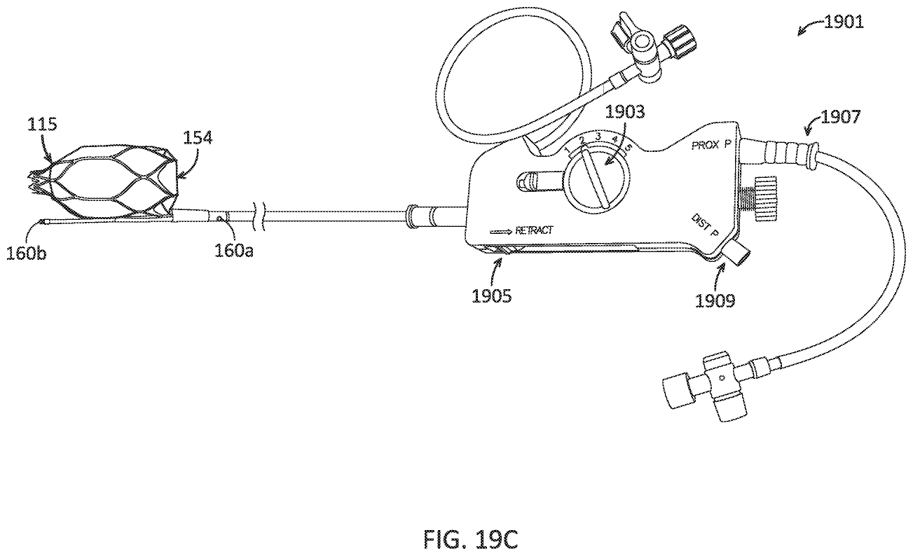

FIGS. 19A-19C illustrate a handle 1901 configured to control delivery and deployment of a flow control device such as the anchor 105 described herein, including control of distal flow control element 115 and proximal flow control element 154. Referring to FIGS. 19A and 19B, the handle 1901 can include a distal flow control element actuator 1903, configured to adjust or control a diameter of the distal end of the anchor by cinching or releasing the distal flow control element. In some embodiments, the actuator 1903 can be stepped or have discrete positions corresponding to specific diameters of or degrees of opening of the distal end of the flow control device. For example, referring to FIG. 19A, the actuator 1903 may have a plurality of positions (e.g., 1 through 5), with a first position (e.g., position 1) corresponding to a widest opening of the distal end of the flow control device (and flow control element) and the last position (e.g., position 5) corresponding to a narrowest opening of the distal end of the flow control device (and flow control element). The intermediate positions can correspond to distal opening diameters between the first and last positions.

Proximal flow control element actuator 1905 can similarly control the size or diameter of the proximal opening of the flow control device and proximal flow control element. In the illustrated example, retracting the actuator 1905 can cause the proximal flow control element to cinch, causing the proximal end of the flow control device to close for easier retraction and removal of the flow control device from the patient.

The handle 1901 can further include ports or lumens 1907 and 1909 corresponding to, for example, the lumens in the catheter 145 used for the proximal and distal pressure sensors described above. A guidewire lumen 1911 is also provided, in addition to one or more saline flush ports 1913.

FIG. 19C is an overall view of the system, showing how actuator 1903 can control the diameter of distal flow control element 115 and actuator 1905 can control the diameter of proximal flow control element 154. Lumen 1907 provides a pathway for proximal pressure sensor 160a (either a fluid column or a lumen for a MEMS sensor wire) and lumen 1909 provides a pathway for distal pressure sensor 160b (either a fluid column or a lumen for a MEMS sensor wire).

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “!”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A blood flow control device comprising:
a catheter adapted to be advanced into a blood vessel to a blood flow control site within the blood vessel;
an anchor supported by the catheter and having an uncompressed configuration and a compressed configuration, the anchor comprising a proximal portion having first diameter in the uncompressed configured and a distal portion having a second diameter in the uncompressed configuration smaller than the first diameter, the proximal portion being adapted to expand toward the uncompressed configuration to engage a wall of the blood vessel, the anchor comprising a blood impermeable wall defining an adjustable blood flow path extending through the anchor from a proximal opening at a proximal end of the proximal portion to a distal opening at a distal end of the distal portion, the catheter being disposed outside of the adjustable blood flow path;
a flow control element supported by the catheter, the flow control element comprising a cinching line adapted to change a shape of the distal portion of the anchor from a first shape to a second shape, thereby changing a size of the distal opening, the cinching line being disposed proximal to the distal end of the distal portion and being adapted to change a rate of blood flow through the blood flow path when the distal portion of the anchor changes from the first shape to the second shape; and
a blood flow control actuator disposed at a proximal section of the catheter and adapted to actuate the cinching line.

2. The device of claim 1, wherein the cinching line extends around an exterior surface of the anchor.

3. The device of claim wherein the cinching line extends through a plurality of supports circumferentially disposed around the distal portion of the anchor.

4. The device of claim 3, wherein each of the plurality of supports comprises a loop extending around a strut of the anchor.

5. The device of claim 1, wherein the distal portion of the anchor is further adapted such that its distal end does not bend radially inward with respect to remaining portions of the distal portion when the distal portion changes from the first shape to the second shape.

6. The device of claim 1, further comprising an anchor distal support adapted to resist movement of the distal portion of the anchor toward the catheter during actuation of the cinching line.

7. The device of claim 1, wherein the anchor comprises a self-expandable scaffold having a plurality of struts, the distal support comprising a distal support strut that is one of the plurality of struts, the distal support strut being stiffer than other struts of the plurality of struts.

8. The device of claim 7, wherein the distal support strut is disposed on a portion of the scaffold facing the catheter.

9. The device of claim wherein the distal support strut comprises at least one loop through which the cinching line extends.

10. The device of claim 1, further comprising a centering device configured to align the anchor with an opening of a sheath to facilitate placement of the anchor in the sheath.

11. The device of claim 10, wherein the anchor is supported by the catheter on an exterior side of the catheter such that the catheter is outside of the anchor, the centering device comprising a ramped surface on the exterior side of the catheter.

12. The device of claim 1, further comprising an anchor collapse control element supported by the catheter and adapted to reduce a cross-sectional dimension of a proximal end of the anchor to facilitate placement of the anchor in the sheath.

13. The device of claim 1, wherein the anchor further comprises a scaffold comprising a plurality of rows of expandable cells defined by struts, cells in a distal row of cells of the plurality of rows of expandable cells having longer struts than cells in a plurality of more proximal rows of cells.

14. The device of claim 13, wherein the cells in the distal row of cells extend over a larger area in the uncompressed configuration than areas over which the cells in the more proximal rows extend in the uncompressed configuration.

15. A blood flow control device comprising:
a catheter adapted to be advanced into a blood vessel to a blood flow control site within the blood vessel;
an anchor supported by the catheter and having an uncompressed configuration and a compressed configuration, the anchor comprising a proximal portion having first diameter in the uncompressed configured and a distal portion having a second diameter in the uncompressed configuration smaller than the first diameter, the proximal portion being adapted to expand toward the uncompressed configuration to engage a wall of the blood vessel, the anchor comprising a blood impermeable wall defining an adjustable blood flow path extending through the anchor from a proximal opening at a proximal end of the proximal portion to a distal opening at a distal end of the distal portion, the catheter being disposed outside of the adjustable blood flow path;
a flow control element supported by the catheter, the flow control element comprising a cinching line adapted to change a shape of the distal portion of the anchor from a first shape to a second shape, the cinching line extending to the anchor from the catheter and being adapted to change a rate of blood flow through the blood flow path when the distal portion of the anchor changes from the first shape to the second shape;
an anchor distal support adapted to resist movement of the distal portion of the anchor toward the catheter during actuation of the cinching line; and
a blood flow control actuator disposed at a proximal section of the catheter and adapted to actuate the cinching line.

16. The device of claim 15, wherein the anchor comprises a self-expandable scaffold having a plurality of struts, the distal support comprising a distal support strut that is one of the plurality of struts, the distal support strut being stiffer than other struts of the plurality of struts.

17. The device of claim 16, wherein the distal support strut is disposed on a portion of the scaffold facing the catheter.

18. The device of claim 16, or wherein the distal support strut comprises at least one loop through which the cinching line extends.

19. The device of claim 15, wherein the distal portion of the anchor is further adapted such that its distal end does not bend radially inward with respect to remaining portions of the distal portion when the distal portion changes from the first shape to the second shape.

20. The device of claim 15, wherein the cinching line is further adapted to change a size of the distal opening when it changes the shape of the distal portion of the anchor.

21-31. (canceled)
